(19) United States
(12) Patent Application Publication
(10) Pub. No.: US 2009/0043232 A1
(43) Pub. Date: Feb. 12, 2009

(54) Sterile Tensor Cravats And Methods For Storing And Deploying Same

(76) Inventor: Anthony G. Borschneck, Redding, CA (US)

Correspondence Address: R. MICHAEL WEST;LAW OFFICES OF R. MICHAEL WEST, 1922 21ST STREET, SACRAMENTO, CA, 95811, US

(21) Appl. No.: 12/255,612

(22) Filed: Oct. 21, 2008

Related U.S. Application Data
(63) Continuation-in-part of application No. 12/009,479, filed on Jan. 18, 2008.
(60) Provisional application No. 60/897,165, filed on Jan. 23, 2007.

(51) Int. Cl. A61F 5/00; A61L 15/00

(52) U.S. Cl. 602/5; 602/36; 602/75

(57) ABSTRACT

An articulated, adjustable, and lockable alignment arm, and associated extensions and accessories, which are user manipulable to model the size and shape of the fractured limb of a patient in the field. The alignment arm is adjusted for proper orientation by setting it over the injury, and then locking the arm into a selected orientation which models the patient's limb around the area of the injury. Next, first and second arm extenders and protective end pads are used to expand the effective size of the alignment arm to form a custom splint structure. Lastly, the splint structure is secured to the patient's injured limb through the use of flexible cravats. The cravats are self-sterilizing, stored so that their sterility is maintained, and deployed so that only the sterile portion of the cravat comes into contact with a patient's skin.

### PRIORITY CLAIM

Pursuant to the provisions of 35 U.S.C. § 119(e)(1), Applicant claims the priority of U.S. Provisional Patent Application Serial No. 60/897,165, filed Jan. 23, 2007.

### CROSS-REFERENCE TO RELATED APPLICATIONS

This application is a continuation-in-part of Ser. No. 12/009,479, filed Jan. 18, 2008.

### BACKGROUND OF THE INVENTION

1. Field of the Invention

The invention relates generally to improvements in apparatus and methods providing medical splinting for fractured or broken human limbs, joints, and body parts. More particularly, the invention pertains to an articulated, adjustable, and lockable alignment arm, and associated extensions and accessories. These components are user manipulable in the field, to create a rigid, padded, structural model which emulates the shape of the fractured limb of a patient.

By securing the structural model and the limb together with fabric tensor cravats, the injured patient may be transported comfortably and safely to a medical center. Each fabric cravat is made and maintained sterile by first impregnating its inner side with a silver-based chemical composition. Then, the inner side is folded back upon itself, followed by folding the outer side back upon itself, until the cravat assumes a compact configuration for storage and eventual deployment in the field in a sterile condition.

2. Description of the Prior Art

Medical splinting apparatus is used in treating and transporting patients who have suffered a bone or joint injury, such as a fracture or dislocation. The task of the splint is to stabilize the broken or fractured body parts quickly and with minimal pain and discomfort to the patient. One recognized problem stems from the wide variety of injuries encountered, ranging from a broken arm or leg, to a dislocated shoulder. Each of these injuries calls for a splint having a different size and configuration. Consequently, the paramedic or doctor must have splints of different sizes and configurations available in the field, so they can be adapted to the patient's injury and used effectively.

Other problems encountered include patient discomfort and possible aggravation of the injury resulting from improper splinting. The splints must be designed so they can be applied with minimal discomfort to the patient, who is likely already in pain. At the same time, an effective splint must stabilize the joint or fracture so that further injury does not occur either during transport or as a consequence of unexpected patient movement during transport to a medical facility.

Where the injury has occurred in the field or under the conditions of a traumatic accident, the job of splinting the injury and transporting the patient to a hospital in a remote location is particularly challenging. The patient may have to be extracted from a damaged motor vehicle, or moved up a steep cliff, even before primary transport has begun. Thus, the splinting apparatus must be lightweight, quick to apply to the patient, and effective in immobilizing the broken or fractured body part.

In U.S. Pat. No. 4,608,971, issued to Borschneck, an emergency leg splint is shown. This device was designed as a single or double leg traction splint for treatment of fractured femurs. This splint worked well clinically. In addition, its use necessitated that the body of the splint extended beyond the patient's feet. This extended length prevented unrestricted use in confined spaces, such as helicopters and ambulances used in patient transport.

There remained a need for a device that would both splint and apply traction to injured limbs, in circumstances where both femurs were fractured. It was also desirable that such a splint would be confined entirely within the lower body profile, to make patient transport easier and safer. Lastly, a review of the anatomy and pathophysiology of fractured femurs and related pelvic structures indicated an improvement was needed in the proximal end of the splint, where it rested against the ischial tuberosity. The ischial tuberosity is located at the distal end of the pelvis, and is distal to the perineal body in both the male and female sexes. These skeletal hard points protect the perineum and form the base platform for the lower torso when a human sits.

To address these needs, an ischial perineal cushion for emergency traction splint was developed. This cushion is shown in U.S. Pat. No. 4,941,465, granted to Borschneck. The cushion, located at the proximal end, seats the splint comfortably and reliably against the ischial perineal protuberances. The distal end of the splint does not extend beyond the lower body profile, facilitating easier transport of the patient. In those respects, the traction splint of the '465 Pat. represented an improvement in the performance and safety of the prior art splints used to stabilize human limbs, joints, and body parts.

Nevertheless, current medical requirements indicate the need for an improved splint device or system that is small, light and compact, for transport to remote and difficult to reach accident locations. There is also a need for a splint which is radiolucent, particularly in the region of the patient's injury. This feature affords the option of leaving the device intact on the patient during X-ray, CAT scan and MRI studies. And, there remains a need for a device which can be configured to splint any fracture of any limb, joint or body part of any size adult or child. More specifically there is a need for a single splint apparatus or system, that can be adapted to accommodate a variety of fractures or dislocations with a minimum of pain and/or movement, and can be applied to the patient's appendages or torso with sterile tensor cravats.

### SUMMARY OF THE INVENTION

The present invention provides for improvements in splints used for transporting injured patients from a field location to a treatment facility. Such improvements include: (1) splints with radiolucent properties around and along the region of the injury, thus allowing X-ray, Cat Scan and MRI imaging of the injury without the need for removing the splint; (2) a portable splint kit which may be packaged, transported, disassembled, or reassembled in the field, quickly and efficiently; (3) splint assemblies which provide both a splinting function and apply a quantifiable and dynamic traction force to one or two broken femurs; (4) a splint modeling system having adjustment and fixation features allowing the splinting of any limb, joint or body part on any human of any size or age; and, (5) fabric tensor cravats for use with the splint assemblies and the splint modeling system, which are maintained in a sterile condition both during storage in the portable splint kit and while being deployed over open wounds on a patient's body.

The heart of the splint modeling system is an articulated, adjustable, and lockable, alignment arm. The alignment arm is comprised of two arm segments, each having one respective end pivotally attached to a respective end of an intermediate connector body. The axes for the pivotal attachments are perpendicular with respect to each other. The arm segments may be independently adjusted and selectively locked into a plurality of rotational positions about a respective axis. In use, the alignment arm is shaped adjacent and parallel to any fractured bone or joint, thereby producing a modeled structure having the same configuration and orientation as the injured limb. The procedure affords accurate splinting of the fractured member without movement or production of pain. Rotating locking knobs on the connector body ensures that the alignment arm retains the shape of the injury. Once the alignment arm is locked into the desired configuration, the arm segments are lengthened through the use of arm extenders fitted with padded material. End pads may also be installed into the free ends of the arm extenders. The assembly is then gently placed on the limb or body, and secured in position with two or more fabric cravats of variable lengths and widths. This affords safe movement of the patient, while securing the injured limbs and joints of the patient in the same position they were found by medical personnel.

The outer surface of the flexible cravats is provided with a dynamic composite elasticized loop fabric, marketed under the trademark BREATH-O-PRENE. The inner surface is provided with an opened cell foam material, embedded with nano-crystals of a silver compound, namely, silver chloride. For the purpose of securing the cravats around the limb and the modeled splint, one end of each cravat includes a short length of hook Velcro sewn thereon. The cravats are folded for storage in a unique manner which completely sterilizes the inner surface to be applied over a patient's open wounds or appendage. When deployed for use, another method of unfolding and applying the cravat, ensures that the sterility of the inner surface is maintained as the cravat is applied to the patient. These sterile cravats create a quick, secure closure member, either for binding the limb to the splint structure, or for restraining or supporting a limb, or for enclosing a wound.

Another application for certain components of the invention is to splint, for example, leg injuries also needing a pre-determined amount of traction. In this application, a bilateral traction splint assembly is provided, including a spring pulley and cable structure, entirely mounted and enclosed within a telescopic housing at the distal end of the assembly. The same arm extenders used in the articulated splint are coupled together to form a straight splint shaft, extending between a base cushion seated against the user's ischial tuberosity at the proximate end of the assembly, and an ankle cravat secured to the user's ankle at the distal end of the assembly. The splint shaft is padded on both lateral surfaces lying along the inner sides of the user's leg, providing enhanced patient comfort.

A carrying case is also provided, housing a kit comprising a disassembled bilateral traction splint, an articulated alignment arm, padded arm extenders, end pads, and a plurality of sterile fabric cravats of varying widths and lengths. In the hands of properly trained medical personnel, this kit is capable of quickly and correctly providing a traction splint, if needed, for fractured femurs, or providing a modeled splint for stabilizing a limb extending from the body of any sized adult or child.

### BRIEF DESCRIPTION OF THE DRAWINGS

FIG. 1 is a side elevational view, showing a prior art leg splint;

FIG. 2 is a perspective view, showing the assembly of various components comprising the portable splint kit of the present invention;

FIG. 3 is a perspective view of the articulated alignment arm;

FIG. 4 is a top plan view of the alignment arm, showing a range of rotation for a first arm segment;

FIG. 5 is a side elevational view of the alignment arm, showing a range of rotation for a second arm segment;

FIG. 6 is an exploded perspective view of the alignment arm, including the first and second arm segments and the intermediate connector means;

FIG. 7 is a perspective view, showing the alignment arm placed on the injured portion of the patient's leg for modeling;

FIG. 8 is an exploded perspective view of the alignment arm being assembled with arm extenders and protective end pads;

FIG. 9 is a perspective view, showing the rotational adjustable feature of an end pad and a first type of coupler means between the pad and the extender;

FIG. 10 is a perspective view, showing the same rotational feature for the other end pad and a second type of coupler means between the pad and the extender;

FIG. 11 is a perspective view showing a typical assembled splint, the phantom representation showing an alternative configuration for the splint;

FIG. 12 is a perspective view showing the splint of FIG. 11 placed over the patient's injured limb;

FIG. 13 is a perspective view, showing the splint of FIG. 12 attached to the patient's limb through the use of a plurality of fabric cravats;

FIG. 14 is a perspective view of an articulated ischio-perineal cushion;

FIG. 15 is a top plan view of a patient with a straight leg injury, such as a dislocated knee, fitted with the field adjustable splint of the present invention;

FIG. 16 is a side elevational view of a patient with a foot or ankle injury, fitted with the splint;

FIG. 17 is a side elevational view of a patient with an elbow or arm injury, fitted with the splint;

FIG. 18 is a perspective view of an open carrying case, displaying the components of the splint kit in a stowed condition;

FIG. 19 is a perspective view showing the first step of folding up the elongated cravat for storage, an inner side of a first length being folded over and in contingent relation with the inner side of a second length, forming a sterile segment, a connector end of the cravat being left exposed;

FIG. 20 is a perspective view showing the second step of folding up the sterile segment of the cravat of FIG. 19 over itself, so two outer side portions will be in contingent relation;

FIG. 21 is a perspective view showing the third step of folding up the sterile segment again partly over itself, to form a more compact package;

FIG. 22 is a perspective view showing the fourth step of folding the connector end over an exposed outer side portion, completing the method of preparing the cravat for storage;

FIG. 23 is a perspective view of a sterile cravat folded up into a compact configuration for storage;

FIG. 24 is a perspective view of a sterile cravat deployed from a storage configuration by detaching the connector end from the outer side, and carefully unfolding and positioning the cravat so that the sterile, inner side is ready for application over a patient's wound;

FIG. 25 is a perspective view, showing the sterile, inner side of the cravat of FIG. 24 partially wrapped around the patient's arm, the arrow indicating the direction of the next wrap over the arm;

FIG. 26 is a perspective view, showing the continued wrapping motion for the cravat, so that only the sterile, inner side will come into contact with the arm;

FIG. 27 shows the completed application of the sterile cravat over a patient's arm, covering the wound;

FIG. 28 is a fragmentary, cross-sectional view taken on the line 28-28 in FIG. 19; and,

FIG. 29 is a fragmentary, cross-sectional view taken on the line 29-29 in FIG. 20.

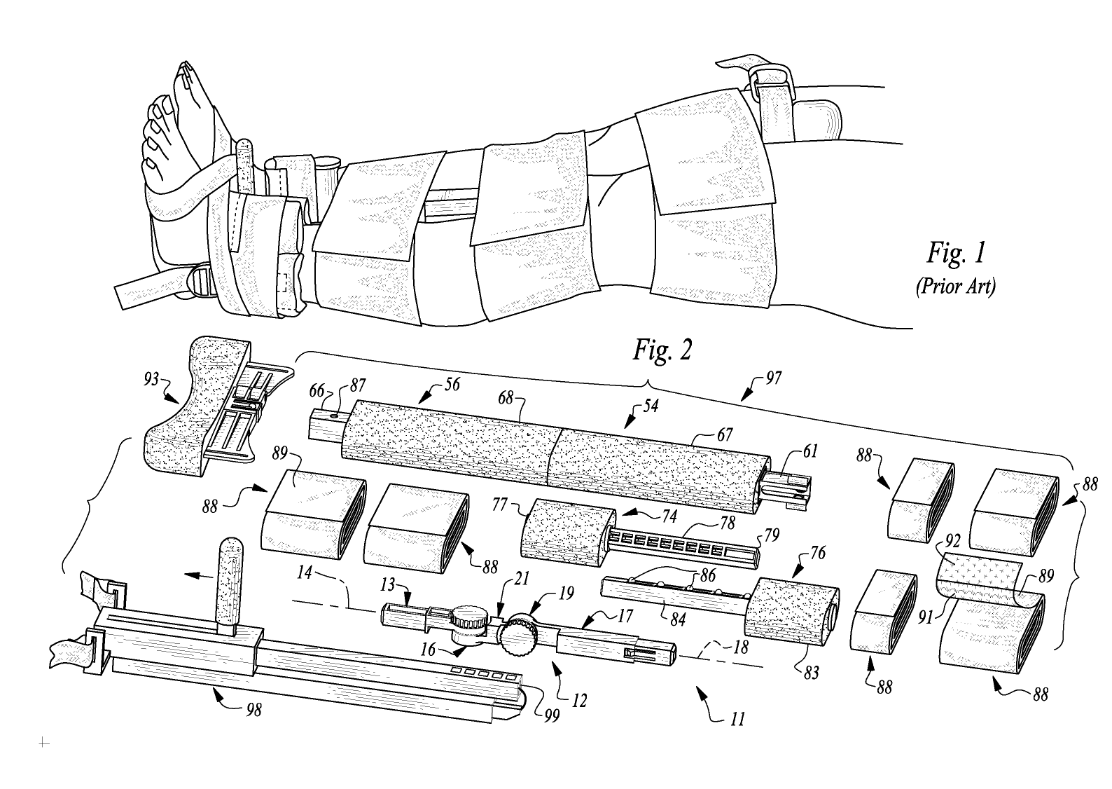

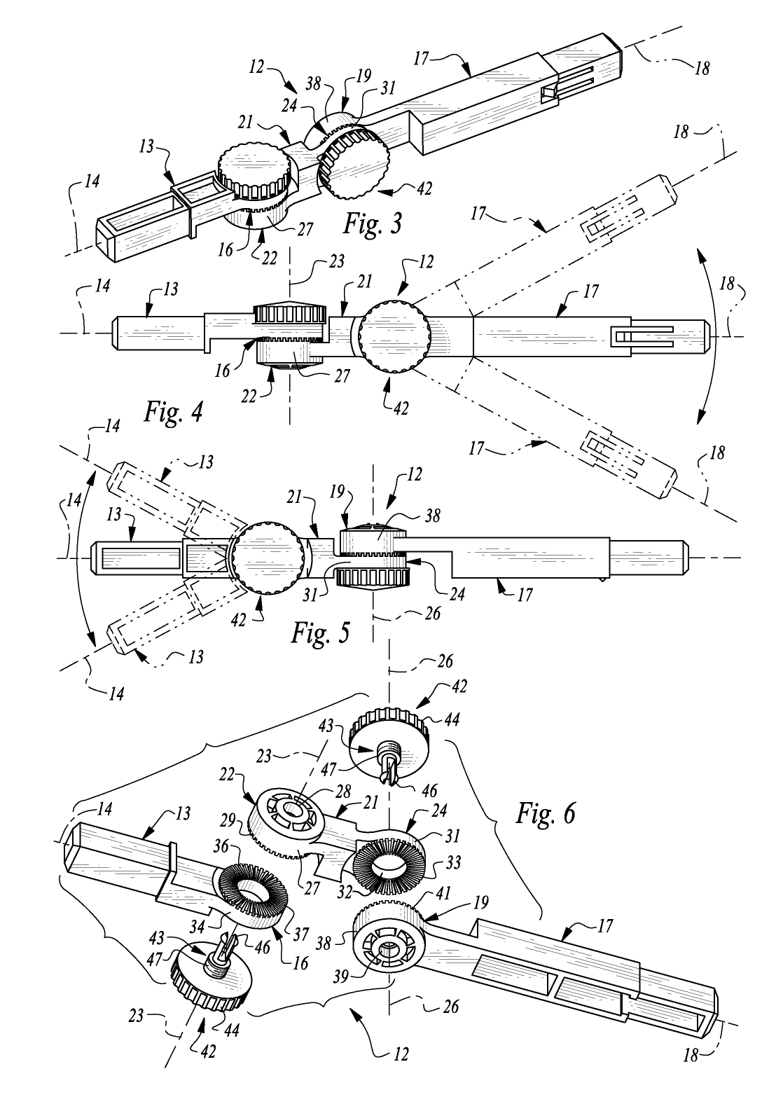

### DETAILED DESCRIPTION OF THE PREFERRED EMBODIMENT

The splint apparatus 11 of the present invention comprises an articulated alignment arm 12, for modeling the orientation and configuration of an injured human limb. Alignment arm 12 includes a first elongated arm segment 13 having a longitudinal axis 14 and a respective pivot end 16, and a second elongated arm segment 17 having a longitudinal axis 18 and a respective pivot end 19. Alignment arm 12 also includes a dual-axis connector body 21 for interconnecting first arm segment 13 to second arm segment 17. Connector body 21 is provided with a first receiver 22 adapted to couple with respective pivot end 16 of first arm segment 13 and lock it at a first selected orientation about a first transverse axis 23. Connector body 21 is also provided with a second receiver 24 adapted to couple with respective pivot end 19 of second arm segment 17 and lock it at a second selected orientation about a second transverse axis 26. As is evident from FIGS. 4, 5, and 6, first transverse axis 23 and second transverse axis 26 are perpendicular with respect to each other, and each transverse axis is also perpendicular to the longitudinal axis of a respective arm segment.

First receiver 22 is provided with a disc portion 27, a first receiver pivot aperture 28, and a plurality of ribs 29 extending radially from the pivot aperture 28. Similarly, second receiver 24 is provided with a disc portion 31, a second receiver pivot aperture 32, and a plurality of ribs 33 extending radially from the pivot aperture 32. For the purpose of coupling with first receiver 22, pivot end 16 of first arm segment 13 includes a respective disc portion 34 provided with a segment pivot aperture 36 and a plurality of ribs 37 extending radially from pivot aperture 36. Similarly, pivot end 19 of second arm segment 17 includes a respective disc portion 38 provided with a segment pivot aperture 39 and a plurality of ribs 41 extending radially from segment pivot aperture 39.

A pair of threaded fasteners 42 is provided for selectively securing the receivers of the connector body in interlocking relation to the pivot ends of the first and second arm segments. Fasteners 42 comprise a shaft 43 having a knob 44 on one end, a resilient barb 46 on the other end, and threads 47 therebetween. Complementary threads are also provided in first receiver pivot aperture 28 and in second arm segment pivot aperture 39.

Fasteners 42 are installed so that the threads in the respective apertures are in a location remote from the knobs 44, so that when the knobs are rotated in clockwise fashion, contingent ribs of the arm segments and the connector body are drawn together in interlocking relation. The location of the threads can easily be reversed, between the pivot apertures in the segments and the connector body, providing identical results and functionality. The barbs 46 act to keep the arm segments and the connector body together, even when the fasteners 42 are completely disengaged from the threads in the apertures. This prevents the loss of parts and assures quick assembly and use of the splint apparatus 11.

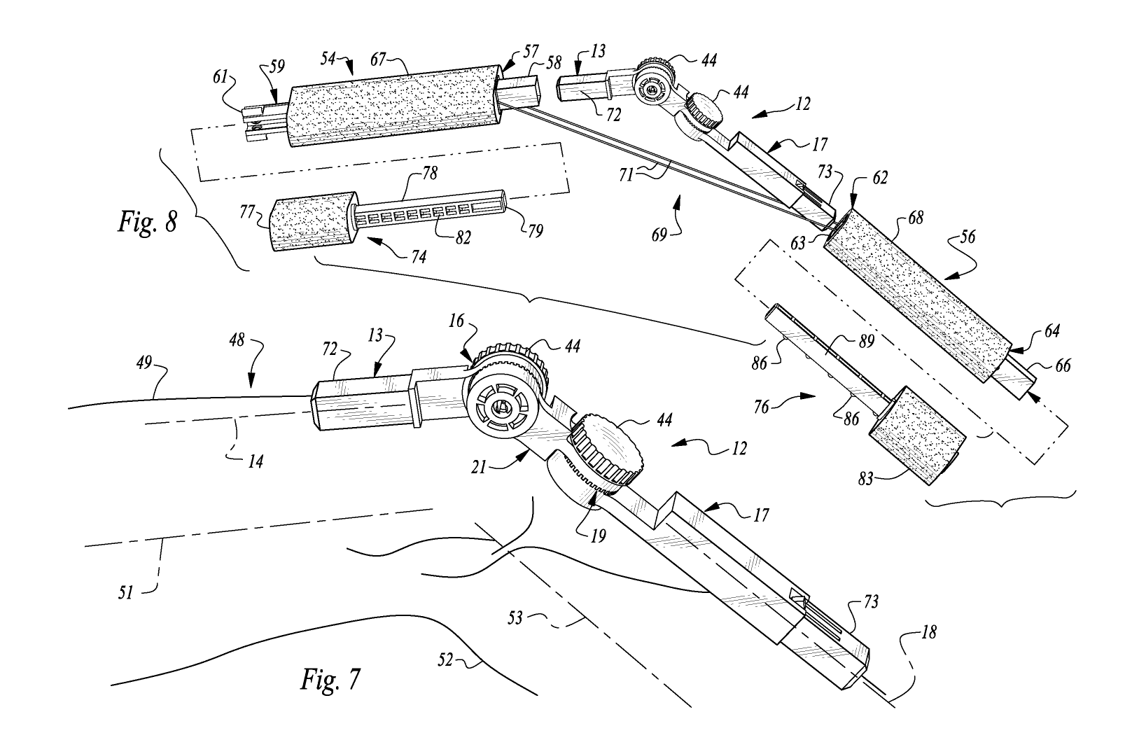

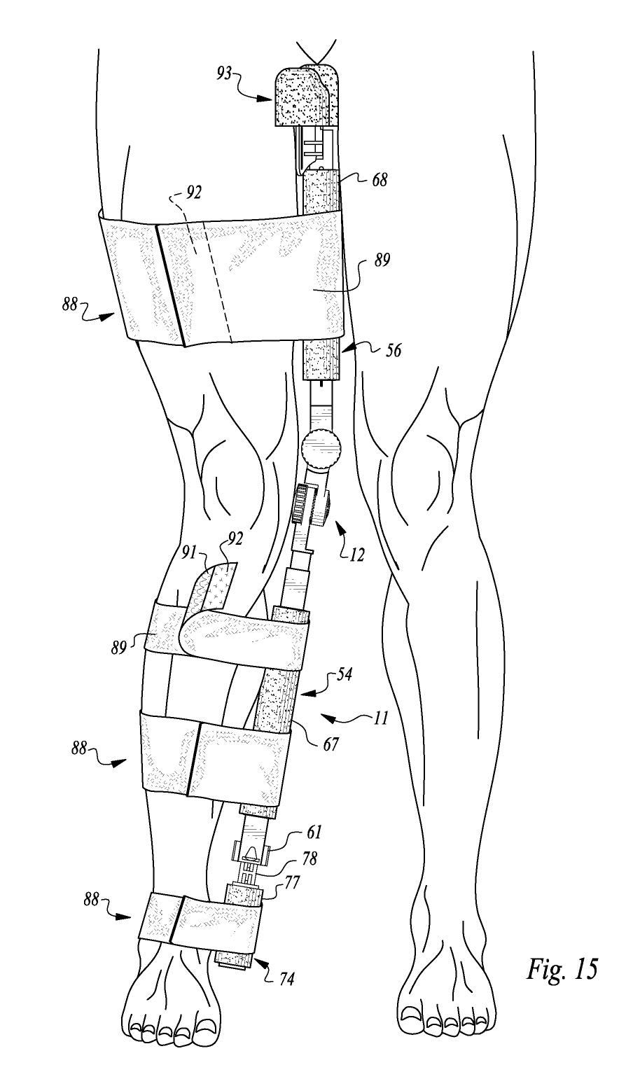

In use of the articulated alignment arm 12, the medical personnel first loosens the knobs 44, so the arm segments 13 and 17 can be pivotally adjusted through a range of motion as generally depicted in FIGS. 4 and 5. The alignment arm is then placed over the critical area of the patient's injured limb or body part. This critical area will vary, depending upon the nature of the injury. For example, in FIG. 7, a bent leg injury such as a bone fracture calls for the alignment arm to be placed generally over the patient's knee cap. In FIG. 15, another bent leg injury such as a dislocated knee requires that the alignment arm be placed along the medial side of the knee cap. FIG. 16 shows the alignment arm 12 in a pre-determined 90° locked orientation, located at the patient's heel for ankle and foot injuries. As yet another example, FIG. 17 shows the alignment arm 12 in a pre-determined 90° locked orientation, located at the patient's elbow, for elbow and lower arm injuries.

Other types of limb and body part injuries which can be accommodated by the splint apparatus include, without limitation, dislocated shoulder injuries, forearm injuries, fracture dislocations of the knee, and straight leg knee injuries. Each of these injuries may call for different orientations and placements for the splint apparatus 11, depending upon the circumstances of the injury and the condition of the limb as the medical personnel finds the patient. Therefore, no attempt will be made to describe each and every potential orientation and placement for the splint apparatus 11, as these will vary widely and the specific applications are within the experience and training of the medical personnel to determine in the field.

Returning to FIG. 7, it is assumed that the injured human limb 48 has a first part 49 with a longitudinal axis 51, and a second part 52 with a longitudinal axis 53. It is further assumed that the human limb 48 has been found by the medical personnel in the same orientation as shown in FIG. 7, upon first encountering the patient. Alignment arm 12 is manually adjusted by the medical personnel so that a first selected orientation places the longitudinal axis 14 of first arm segment 13 in generally parallel relation to the longitudinal axis 51 of first part 49. Then, alignment arm 12 is manually adjusted so that a second selected orientation places the longitudinal axis 18 of second arm segment 17 and the longitudinal axis 53 of the second part 52 in generally parallel relation. Lastly, knobs 44 are both rotated clockwise to secure and lock the alignment arm in an orientation which emulates the configuration of the injured limb 48, in and around the critical area where the first part and the second part join. Now that the alignment arm has a fixed configuration, the rest of the splint apparatus 11 can be assembled and then secured to the injured limb.

For the purpose of expanding the physical size of the alignment arm so it can become a useful splint, a first arm extender 54 and a second arm extender 56 are provided. First arm extender 54 has one end 57 provided with a coupler 58 and another end 59 provided with a coupler 61. Second arm extender 56 has one end 62 provided with a coupler 63 and another end 64 provided with a coupler 66. Foam padding 67 is provided entirely around and along arm extender 54, between couplers 58 and 61. Similarly, foam padding 68 is provided entirely around and along arm extender 56, between couplers 63 and 66.

Resilient and flexible bias means 69, for drawing arm extenders 54 and 56 together, interconnects one end 57 with one end 62. Bias means 69 is preferably comprised of two stretchable cords 71 being stretchable to the extent shown in FIG. 8. With the alignment arm removed from the injured limb, coupler 58 on one end 57 of first arm extender 54 is detachably coupled to an end fitting 72, located on first arm segment 13 remote from pivot end 16. The inner size and configuration of coupler 58 is such that it slides over fitting 72. Stretching out bias means 69 as shown in FIG. 8, coupler 63 on one end 62 of second arm extender 56 is detachably coupled to an end fitting 73, located on second arm segment 17 remote from pivot end 19. Coupler 58 and fitting 72 and coupler 63 and fitting 73, are preferably sized so they will only mate with each other, so that the alignment arm 12 and the arm extenders 54 and 56, can only be assembled one way. The cords 71 of resilient bias means 69 are dressed around knobs 44, ensuring a clean splint structure as the arm extenders are pulled into engagement with alignment arm 12. (See, FIG. 11).

Other features designed to extend the length and adaptability of the splint apparatus 11 include a first end pad 74 and a second end pad 76. First end pad 74 includes a foam padded portion 77, a shaft 78, and an end coupler 79. Coupler 61 includes a resiliently biased finger 81 which engages apertures 82 to secure end pad 74 to the splint assembly 11 in a selected longitudinal position. First end pad 74 is thereby adapted, adjustably and detachably, to interconnect with coupler 61 on first arm extender 54. Second end pad 76 includes a foam padded portion 83, a shaft 84, and a plurality of resiliently biased protuberances 86 spaced along shaft 84. Coupler 66 includes an aperture 87 sized and located for engagement by protuberances 86. Second end pad 76 is thereby adapted, adjustably and detachably, to interconnect with coupler 66 on second arm extender 56 at a selected longitudinal location at the end of splint apparatus 11.

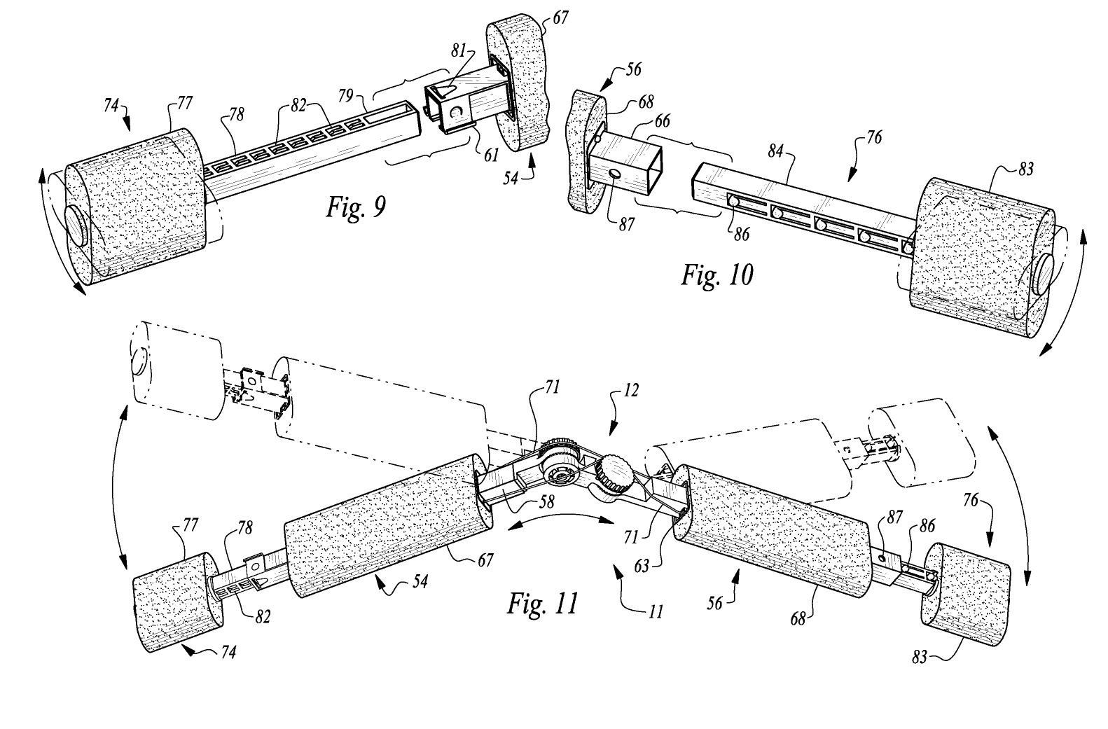

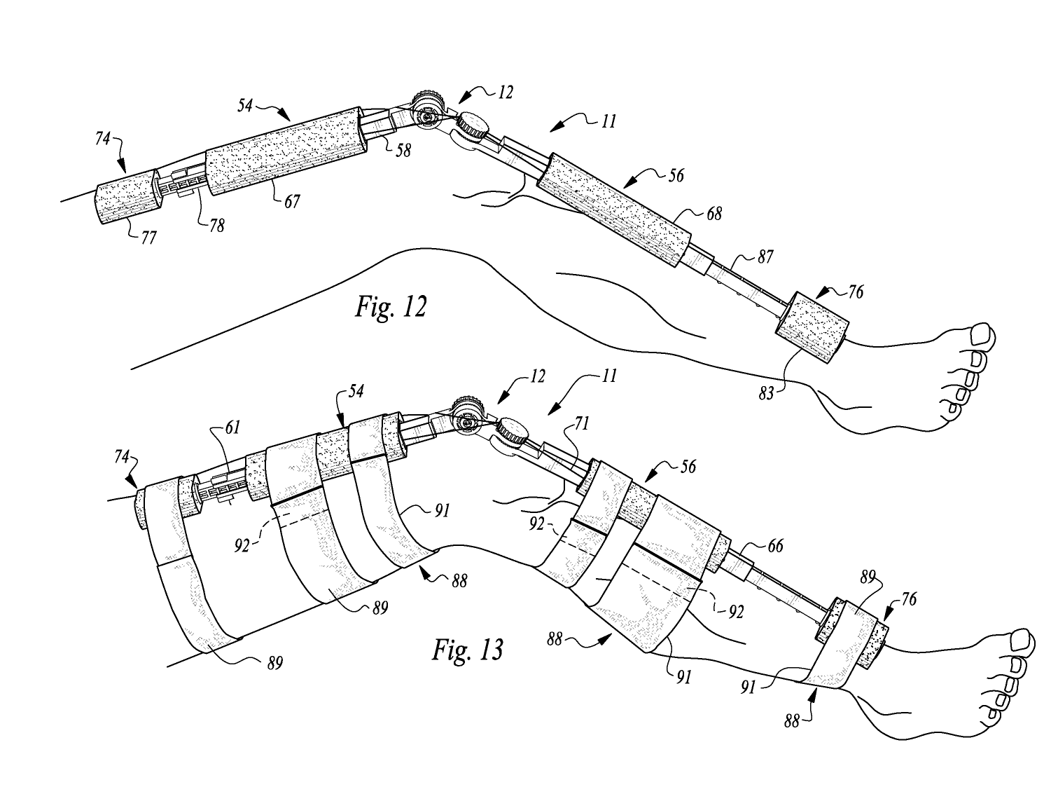

First end pad 74 and second end pad 76, both include means for rotational adjustment of their respective foam padded portions 77 and 83. As shown in FIGS. 9 and 10, foam padded portions 77 and 83 may be rotated into a selected location about the axis of their respective supporting shafts. This feature allows the medical personnel to adjust these end pads into a location which will be most effective and comfortable, when the splint apparatus is fitted to the patient.

The resulting custom fitted splint apparatus 11 is carefully lowered over the patient, and arranged in alignment with the injured limb. The medical personnel then employs a plurality of flexible, resilient, and adjustable cravats 88, having different lengths and widths. These cravats are passed over and around the splint apparatus 11 and adjacent portions of the patient's body, above and below the injured critical area, to secure the splint to the patient. The outer surface 89 of the cravats is provided with a dynamic composite elasticized loop fabric, marketed under the trademark BREATH-O-PRENE™. The inner surface 91 is provided with an opened cell foam material, embedded with nano-crystals of silver chloride to provide bacteriostatic and bactericidal qualities to the cravats. For the purpose of securing the cravats around the limb and the modeled splint apparatus 11, one end of each cravat 88 includes a short strip 92 of hook VELCRO® sewn thereon. These cravats 88 create a quick, secure closure member, binding the injured limb to the rigid structure of the splint apparatus 11.

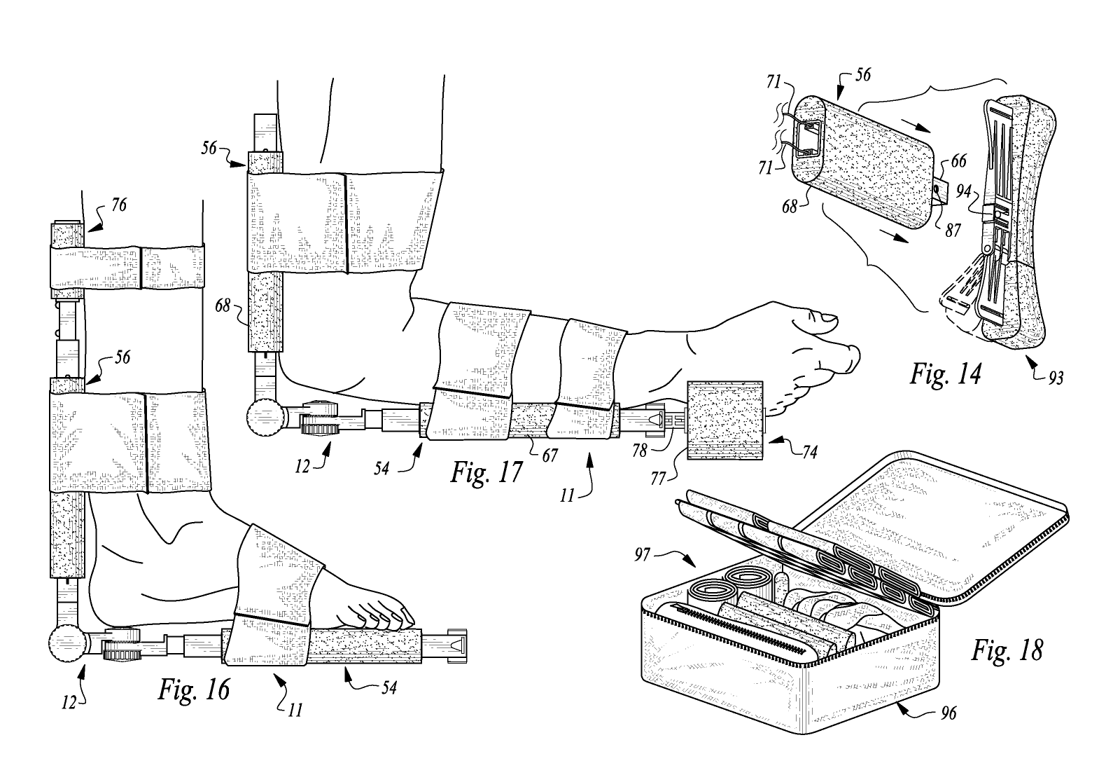

Treatment of a straight knee injury or a dislocated knee injury may call for a special adaptation of the splint apparatus 11. For example, in FIG. 15, the splint apparatus has been fitted on one end with an ischio-perineal cushion 93, including a receiver 94. As shown in FIG. 14, coupler 66 is inserted into receiver 94, thereby securing the cushion 93 to the splint apparatus. Cushion 93 includes a hinged, adjustability feature, described in more detail in U.S. Pat. No. 4,941,465, hereby incorporated by reference as if fully set forth herein. The medical personnel applies the modified apparatus to the patient, seating the ischio-perineal cushion against the ischio-perineal protuberances of the pelvis. Cravats 88 are selectively applied around the splint apparatus 11 and the patient's leg, both above and below the knee injury.

It is also possible to eliminate the use of the alignment arm 12 altogether, by simply sliding first arm extender 54 into second arm extender 56. This can be done easily, as couplers 58 and 63 are sized and configured to mate. This mated configuration is shown in FIG. 2. This arrangement, in combination with the ischio-perineal cushion 93 on one end of the splint, and a first end pad 74 on the other end of the splint, would be appropriate when it is desired to have an entirely straight splint apparatus.

It should also be noted that with the exception of special applications requiring use of the ischio-perineal cushion 93, the splint apparatus 11 is end-to-end reversible. In other words, the splint can be used by placing the second arm extender 56 and the second end pad 76 in a proximate relationship with respect to the patient's body, as shown in FIGS. 16 and 17, or in a distal relationship with respect to the patient's body, as shown in FIG. 13.

The portable splint apparatus 11 of the present invention is preferably carried in a cordura case 96. Case 96 is capable of housing as a kit 97, all of the components necessary to make a wide variety of splint structures in a compact and easily accessible manner. In addition to the components discussed above, the kit 97 may also include a traction device 98, for applying a predetermined amount of pulling tension to the splint. For that purpose, traction device 98 includes a coupler 99 on its end, adapted to engage with coupler 61 on the end of first arm extension 54. In other words, the second end pad is replaced with the traction device 98. In addition, such an arrangement would require a straight line splint, and would not rely upon use of the alignment arm 12. Typically, this arrangement may be employed where the patient's injury is a fractured femur.

It has been determined that by folding the cravats 88 into a compact configuration for storage using a particular method, the nano crystals embedded in the inner surface 91 will begin to kill all microorganisms therein. Using this method, the inner surface of the folded cravat is essentially made sterile within 30 minutes. And, providing the cravat 88 is carefully deployed for use employing the reverse of the folding method, the sterilized inner surface 91 of the cravat may be applied directly upon the wounded limb or body of the patient, substantially reducing the chance of infection.

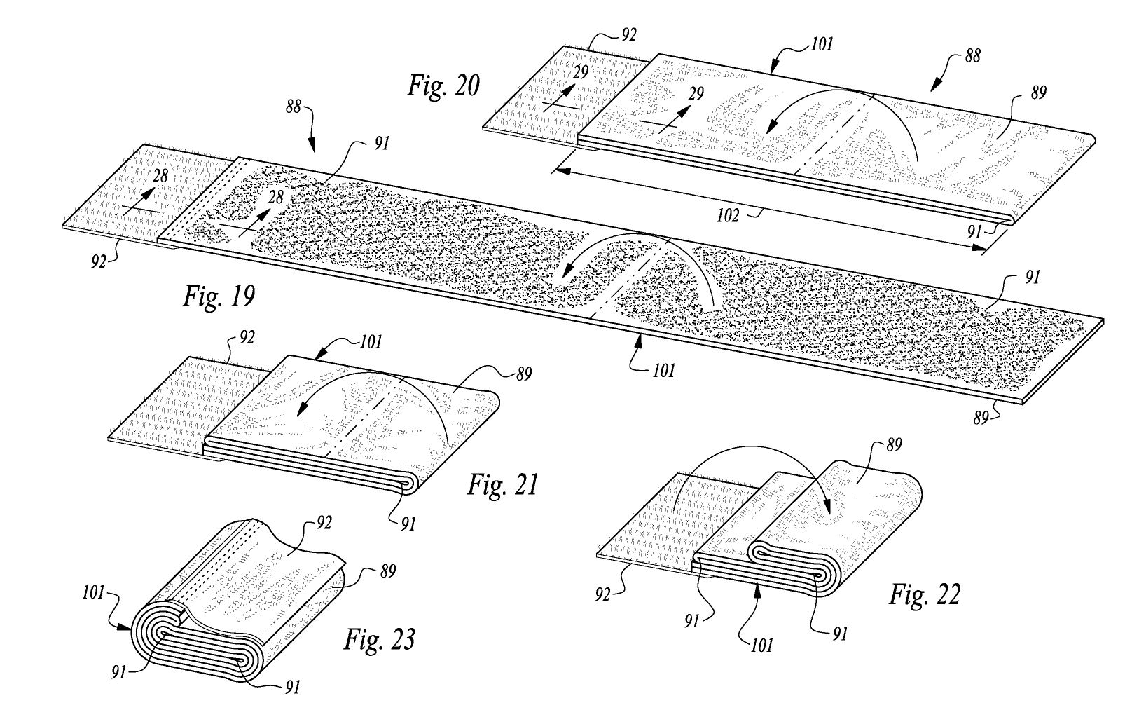

Making particular reference to FIG. 19, the cravat 88 comprises a band 101 having an outer surface 89 and an inner surface 91. The outer surface 89 of the cravat 88 is comprised of a dynamic composite elasticized loop fabric, marketed under the trademark BREATH-O-PRENE™. This elasticized fabric includes an open cell structure, allowing air and moisture to pass relatively freely therethrough. Such a material facilitates cooling of the skin, while allowing moisture to escape from the wound area. The inner surface 91 is provided with an open cell foam material, impregnated with nano-crystals of silver chloride (AgCl). This provides bacteriostatic and bactericidal qualities to the inner surface of the band 101. BREATHE-O-PRENE provided with this foam layer of embedded silver chloride nanocrystals on one side is marketed under the trademark SILVERtec.

In practicing the method of storing such a flexible tensor cravat so that it is maintained in a sterile condition, the inner side 91 of band 101 is first folded back over itself to form a sterile cravat segment 102. In this condition, only the outer surface 89 of the cravat 88 is exposed, and the inner surface 91 of the band is in a sealed, contingent relation with itself. This relationship is best illustrated making reference to FIGS. 20 and 29. The silver chloride nanocrystals immediately work to kill all bacteria which may exist in the inner surface 91, essentially rendering it sterile in a number of hours. It is apparent that the sterile cravat segment 102 could also be rolled up upon itself and enjoy the same beneficial result. However, this second method results in a less compact configuration.

Of course, the length of sterile cravat segment 102 will depend upon the initial length of the band 101. Since cravats of varying lengths may be required for different applications, the number of successive steps required to complete the method and prepare the cravat for storage will vary depending upon the cravat length. In any event, the method herein contemplates that the sterile cravat segment 102 is successively folded back over itself, as shown in FIGS. 21 and 22, until it assumes a storage configuration as shown in FIG. 23. At that juncture, the strip 92 having hook material is extended over an underlying portion of the outer side 89 of cravat 88, having loop material. Using such connector means, the band 101 is maintained in a compact configuration for storage.

When it becomes necessary to deploy the cravat 88 for use on an appendage or body portion of a patient, the method set forth above is reversed, carefully maintaining the sterile inner surface of the cravat 88 in a sterile condition. To that end, the strip 92 is first removed from the underlying portion of the outer side 89 of the cravat 88. The cravat is then unfolded, reversing the steps shown in FIGS. 22, 21 and 20. This is most easily accomplished by grasping the strip 92 at the end of the band 101 and holding it in a substantially fixed relation. Then, the sterile cravat segment 102 is successively unfolded from a stored configuration until it assumes the configuration shown in FIG. 20, with the inner side 91 still folded upon itself in contingent relation.

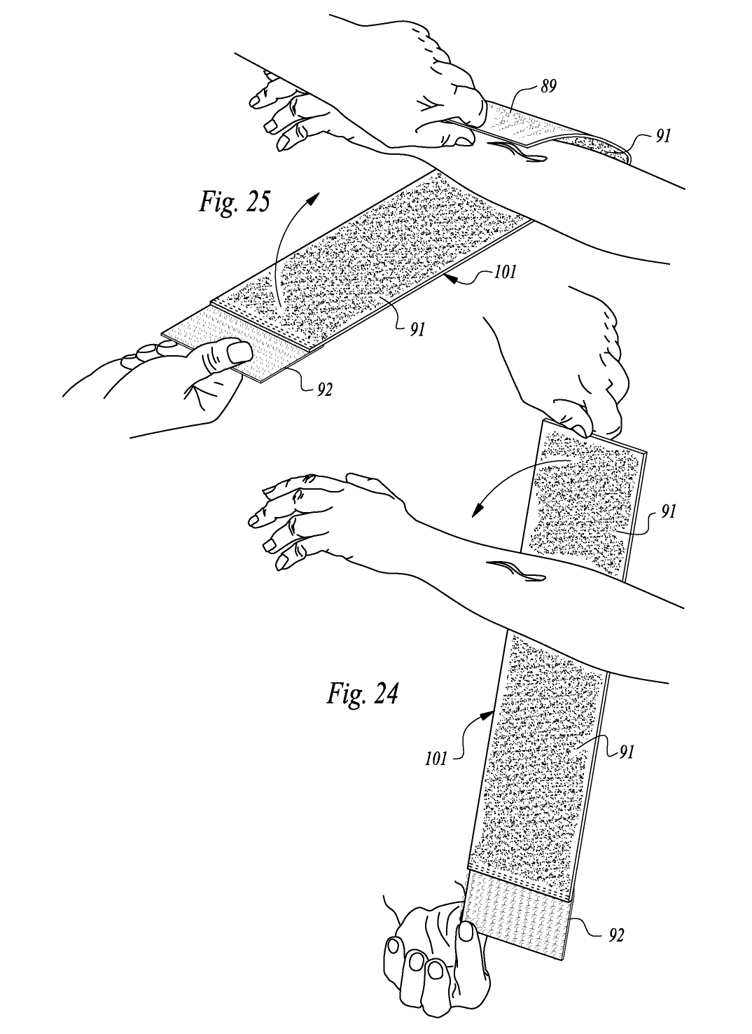

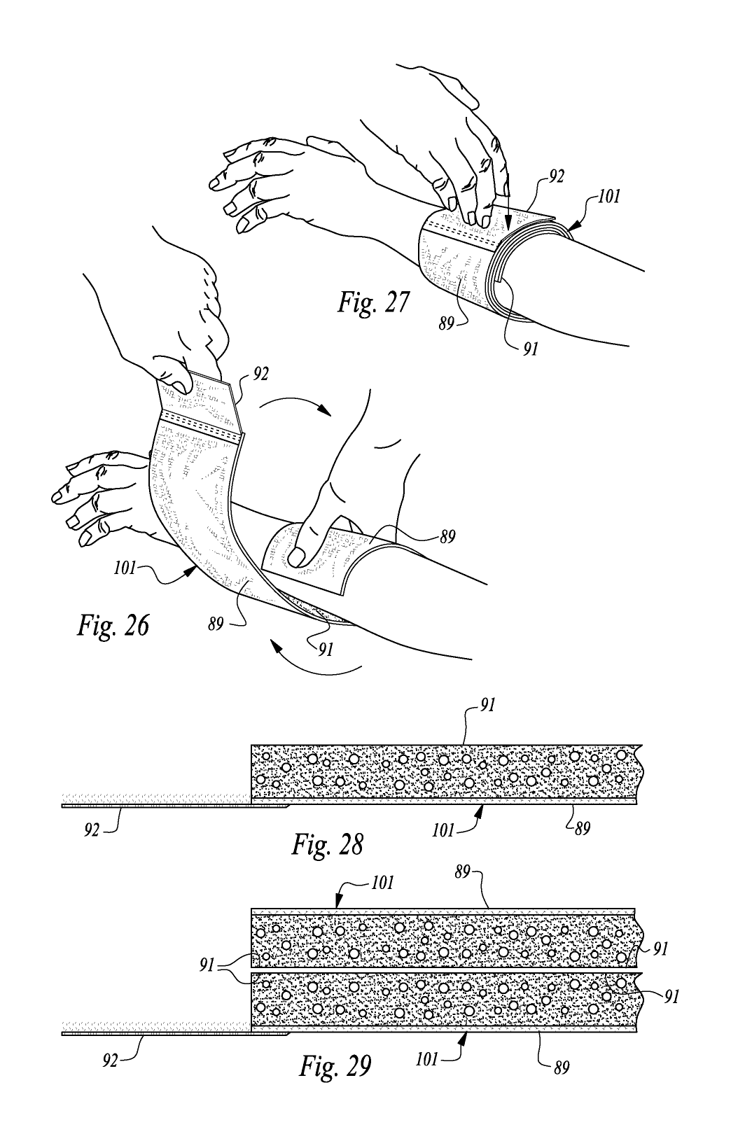

While grasping the strip end with one hand and grasping the other end of the band 101 with the other hand, the sterile cravat segment 102 maybe pulled apart, extending the cravat to its full length. Then, as shown in FIG. 24, the medical technician places a portion of sterile inner side 91 over a patient's arm, and continuously wraps the inner side of said band around the patient's appendage over the outer side 89 of the band until the full length of the band is wrapped up. (See, FIGS. 25-26). Lastly, the strip 92 is folded over and connected to the outer side 89 of the band, making the installation of the cravat 88 complete.

Employing this method of storage and deployment, cravats 88 of different lengths and widths, as appropriate, may be used to bind patients wounds over sterile dressings or to strap fractured limbs to splints. By stretching the fabric of the band 101, pressure is applied to the wound or limb as required, securing the limb in place and helping to control any bleeding. Different sizes and thicknesses and lengths of the cravats 88 provided with the hook and loop connector system, allow these cravats to be used as bandage usable in a wet environment. The cravats 88 may also be washed, dried, and reused up to 30 times, without losing the ability to self sterilize using the bactericidal and bacterostatic properties of the band 101.

It will be appreciated, then, that I have disclosed an articulated alignment arm which is used in conjunction with associated extensions and accessories, to create a custom splint apparatus. This splint assumes the exact configuration and orientation of the injured limb in the position found by medical personnel. The splint apparatus fits closely to the silhouette of the fractured limb and becomes an excellent tool for removing injured patients from confined spaces. The splint apparatus disclosed herein will fit any adult or child, any size, any weight, and can be used to splint any limb injury, including fracture dislocations of the shoulder, elbow, knee and ankle.

It will be appreciated that I have also disclosed a method for folding up cravats into a compact configuration for storage, in which bactericidal and bacterostatic properties of the inner surface of the cravat will kill all microorganisms therein in short order. For later deployment and use of the cravat, the method disclosed herein is reversed, and the sterilized inner surface of the cravat is applied directly upon the wounded limb or body of the patient, substantially reducing the chance of infection.

What is claimed is:

1. A flexible tensor cravat, comprising an elongated band of elasticized fabric having an inner side and an outer side, one end of said band including means for detachably connecting said one end to said outer side of said band, said inner side of said band being impregnated with a bactericidal and bacterostatic compound, said band being folded into a storage configuration with said inner side first being folded back over itself to form a sterile cravat segment, said outer side of said cravat segment then being successively folded back over itself to assume said storage configuration, said connector means then being extended over and attached to an underlying portion of said outer side, to maintain said band in said storage configuration.

2. A cravat as in claim 1 in which said bactericidal and bacterostatic compound comprises nano crystal of silver chloride.

3. A cravat as in claim 1 in which said connector means comprises a strip of hook material from a hook and loop fastening system extending from said one end of said band.

4. A cravat as in claim 1 in which said inner side of said band is coated with open cell plastic foam.

5. A cravat as in claim 1 in which said outer side of said band comprises hook fabric.

6. A method of storing a flexible tensor cravat, comprising the steps of:
a. providing an elongated band of elasticized fabric having an inner side and an outer side, one end of said band including means for detachably connecting said one end to said outer side of said band, said inner side of said band being impregnated with a bactericidal and bacterostatic compound;
b. folding said inner side back over itself to form a sterile cravat segment;
c. successively folding said sterile cravat segment back over itself until it assumes a storage configuration;
d. extending said connector means over an underlying portion of said outer side and attaching said connector means to said underlying portion, thereby maintaining said band in said storage configuration.

7. A method as in claim 6 in which said bactericidal and bacterostatic compound comprises nano crystal of silver chloride.

8. A method as in claim 6 in which said connector means comprises a strip of hook material from a hook and loop fastening system extending from said one end of said band.

9. A method as in claim 6 in which said inner side of said band is coated with open cell plastic foam.

10. A cravat as in claim 6 in which said outer side of said band comprises hook fabric.

11. A method for deploying a flexible tensor cravat from a storage configuration, said cravat including an elongated band of elasticized fabric having an inner side and an outer side, one end of said band including means for detachably connecting said one end to said outer side of said band, said inner side of said band being impregnated with a bactericidal and bacterostatic compound, said band being folded into said storage configuration with said inner side first being folded back over itself to form a sterile cravat segment, said outer side of said cravat segment then being successively folded back over itself to assume said storage configuration, said connector means then being extended over and attached to an underlying portion of said outer side, to maintain said band in said storage configuration, comprising the steps of:
a. removing said connector means from said underlying portion of said outer side;
b. maintaining said one end of said band in substantially fixed relation, while successively unfolding said sterile cravat segment from said storage configuration until it is unfolded to the extent that said inner sides remain in contingent relation;
c. grasping said one end with one hand and grasping said other end with the other hand, and pulling said cravat segment apart and extending said band to a full length;
d. placing a portion of said inner side of said other end of said band over a patient's body;
e. continuously wrapping said inner side of said band around the patient's body and over said outer side of said band until said full length of said band is wrapped up;
f. connecting said one end to said outer side of said band.

12. A method as in claim 11 in which said bactericidal and bacterostatic compound comprises nano crystals of silver chloride.

13. A method as in claim 11 in which said connector means comprises a strip of hook material from a hook and loop fastening system extending from said one end of said band.

14. A method as in claim 11 in which said inner side of said band is coated with open cell plastic foam.

15. A cravat as in claim 11 in which said outer side of said band comprises hook fabric.

16. A method for creating and applying a splint structure which models the configuration of an injured patient's limb, comprising the steps of:
a. providing an articulated, adjustable, and lockable alignment arm, said alignment arm having a connector body with ends pivotally attached to one end of first and second arm segments, said arm segments being rotationally adjustable and lockable into selected rotational positions about respective perpendicular pivotal axes;
b. locating said alignment arm over the injured limb of the patient;
c. adjusting said alignment arm to form a structural model which emulates the shape of the injured limb of the patient;
d. locking said first and second arm segments of said structural model into respective fixed relations with respect to said connector body;
e. coupling first and second arm extenders with the other ends of said first and second arm segments;
f. positioning said alignment arm fitted with said first and second arm extenders over the injured limb of the patient;
g. deploying a flexible tensor cravat from a storage configuration, said cravat including an elongated band of elasticized fabric having an inner side and an outer side, one end of said band including means for detachably connecting said one end to said outer side of said band, said inner side of said band being impregnated with a bactericidal and bacterostatic compound, said band being folded into said storage configuration with said inner side first being folded back over itself to form a sterile cravat segment, said outer side of said cravat segment then being successively folded back over itself to assume said storage configuration, said connector means then being extended over and attached to an underlying portion of said outer side, to maintain said band in said storage configuration;
h. removing said connector means from said underlying portion of said outer side;
i. maintaining said one end of said band in substantially fixed relation, while successively unfolding said sterile cravat segment from said storage configuration until it is unfolded to the extent that said inner sides remain in contingent relation;
j. grasping said one end with one hand and grasping said other end with the other hand, and pulling said cravat segment apart and extending said band to a full length;
k. placing a portion of said inner side of said other end of said band over an underlying portion of the injured limb of a patient;
l. continuously wrapping said inner side of said band completely around said first and second arm extenders and the injured limb of the patient, securing and applying the splint structure to the limb, and over said outer side of said band until said full length of said band is wrapped up;
m. connecting said one end to said outer side of said band.

17. A method as in claim 16 in which the injured limb of the patient is a leg.

18. A method as in claim 16 in which the injured limb of the patient is an arm.

19. A method as in claim 16 in which the injured limb of the patient is a foot.

20. A method as in claim 16 in which the injured limb is a leg and the injury is to the knee of the leg.
